(19) United States
(12) Patent Application Publication
(10) Pub. No.: US 2017/0032702 A1
(43) Pub. Date: Feb. 2, 2017

(54) Method and Apparatus For Generating an Ultrasound Scatterer Representation

(71) Applicant: EIDGENOSSISCHE TECHNISCHE HOCHSCHULE ZURICH, Zurich (CH)

(72) Inventors: Orcun GOKSEL, Zurich (CH); Oliver MATTAUSCH, Zurich (CH)

(21) Appl. No.: 15/218,716

(22) Filed: Jul. 25, 2016

Related U.S. Application Data
(60) Provisional application No. 62/309,298, filed on Mar. 16, 2016.
(60) Provisional application No. 62/197,102, filed on Jul. 27, 2015.

(51) Int. Cl. G09B 23/28; G06T 7/00; G06T 11/00; A61B 8/00

(52) U.S. Cl. CPC G09B 23/286; A61B 8/00; G06T 7/0012; G06T 11/003; G06T 2207/10132

(57) ABSTRACT

Realistic ultrasound imaging simulation requires modeling of scatterers corresponding to different imaging speckle appearances. A scatterer generator acquires a plurality of ultrasound signal samples, each corresponding to a different ultrasound capture and reconstructs a scatterer representation from the ultrasound signal samples and associated Point Spread Functions. Point Spread Functions (PSFs) may be estimated from multiple image acquisitions at the same reference position resulting from beam-steering. Reconstructed scatterers may then directly be used in ultrasound simulation or an additional step of modeling the scatterers may be applied. Statistical distribution parametrization or texture synthesis may be used to model the scatterers. Different scatterer models may be used for different homogeneous regions. The reconstructed scatterers and/or the scatterer models may be registered into a library of scatterers by the scatterer generator.

### CROSS-REFERENCE TO RELATED APPLICATIONS

This application claims the benefit of U.S. Provisional Patent Application Ser. No. 62/197,102, filed Jul. 27, 2015, naming Orcun Goksel, Oliver Mattausch, entitled “Method and apparatus for generating an ultrasound scatterer representation”, and of U.S. Provisional Patent Application Ser. No. 62/309,298, filed Mar. 16, 2016, naming Orcun Goksel, Oliver Mattausch, entitled “Method and apparatus for generating an ultrasound scatterer representation”, which are herein incorporated by reference in their entirety for all purposes.

### FIELD OF THE INVENTION

Methods and apparatuses described herein relate to ultrasound image simulation in general, and more specifically to virtual reality ultrasound image modeling for ultrasound practice training simulation purposes, for example in the medical field.

### BACKGROUND

### Medical Imaging Simulators

Application of ultrasound requires a high level of expertise both in manipulating imaging devices and also in analyzing and interpreting resulting images, for instance in the medical field for accurate diagnosis and intervention guidance. Learning the proper execution of this modality thus requires a lengthy training period for ultrasound specialists.

To facilitate the training of medical students and doctors, advanced medical procedure simulators may be used such as the one described in U.S. Pat. No. 8,992,230. Such simulators may be based on a virtual reality (“VR”) and/or a mixed or augmented reality (“AR”) simulation apparatus, by which the physician may experiment a medical procedure scenario. The VR/AR system may compute and display a visual VR/AR model of anatomical structures in accordance with physician gestures and actions to provide various feedback, such as visual feedback. In a VR system, an entire image may be simulated for display to a user, and in an AR system, a simulated image may be overlaid or otherwise incorporated with an actual image for display to a user. Various patient models with different pathologies can be selected. Therefore, natural variations as encountered over the years by practicing medical staff can be simulated for a user over a compressed period of time for training purposes.

### Ultrasound Imaging Simulation

Early ultrasound simulation solutions have been developed based on interpolative ultrasound simulation, such as for instance the method developed by O. Goksel and S. E. Salcudean as described in “B-mode ultrasound image simulation in deformable 3-D medium”, IEEE Trans Medical Imaging, 28(11):1657-69, November 2009 [Goksel2009]. Interpolative approaches can generally generate realistic images, but only in the absence of directional image artifacts and for images from limited fields of view. In order to handle different field-of-views and to better simulate certain artifacts, as required by certain ultrasound applications such as abdominal ultrasound, other approaches are needed.

### Generative Simulation

Generative simulation, such as wave-based or ray-based ultrasound simulation, aims at emulating the ultrasonic signal that would be registered by a transducer position/orientation, using a combination of analytical and numerical solutions in real-time. However, simulating all possible ultrasound-tissue interaction phenomena is still an unsolved theoretical problem. For instance, the ultrasound texture (speckles) is a result of constructive and destructive interference of ultrasonic waves mainly scattered by sub-wavelength particles, such as cell nuclei, other organelles, etc. However, no known method can observe a sufficiently large tissue region (40-150 mm for OB/GYN ultrasound examination) in such fine detail with cellular structures.

Various methods with trade-offs and approximations to different wave characteristics have been proposed in the literature:

Wave-based Generative Simulation. The wave nature of ultrasound becomes particularly important during its interaction with sub-wavelength particles, which causes part of the incident ultrasound to scatter. Since this is a major source of the speckled texture characteristic to ultrasound, the simulation of scattering effect received significant interest over the decades. In J. A. Jensen, Field: A program for simulating ultrasound systems, In 10th North-Baltic Conf on Biomedical Imaging, pages 351-353, 1996 [Jen96], Jensen proposed an acoustic model for computing the ultrasonic Point-Spread Function (PSF), which is the spatial distribution of sound pressure and equivalently the (impulse) ultrasound response that will theoretically be received from a single idealized scatterer. Then, realistic scattering patterns for homogenous medium can be generated using randomly distributed scatterers (approximately 10-1000 mm⁻³) using space-variant PSFs, which can also take into account complex transducer element geometries and characteristics. Nevertheless, due to the sheer number of scatterers involved, this can take several hours per frame computation time, which is impractical and irrelevant for real-time simulation. Such accuracy is often only needed for evaluating the ultrasonic field of a new transducer design, giving the public release of [Jen96]'s simulation its name FieldII, that is also one of the most cited simulation methods in the literature. An alternative technique is the linearized wave model as proposed by Bamber and Dickinson in J C Bamber and R J Dickinson, Ultrasonic b-scanning: a computer simulation, Phys Med Biol, 25(3):463-479, 1980 [BD80] to approximate PSF, using which the response from a single scatterer can be computed by a convolution of few simple separable functions. [BD80] first demonstrated this in 2D planar homogenous domains. A fast convolution extension speeding this method up to 30 s/frame was later proposed by Gao et al in Hang Gao, Hon Fai Choi, P Claus, S. Boonen, S. Jaecques, G. H. van Lenthe, G. Van Der Perre, W Lauriks, and J. D'hooge, A fast convolution-based methodology to simulate 2-D/3-D cardiac ultrasound images, IEEE Trans UFFC, 56(2): 404-409, 2009 [GCC+09]. Fast discrete convolution in image sequences with moving scatterers and blood flow was introduced in Adrien Marion and Didier Vray, Toward a real-time simulation of ultrasound image sequences based on a 3-d set of moving scatterers, Ultrasonics, Ferroelectrics and Frequency Control, IEEE Transactions on, 56(10): 2167-2179, 2009 [MV09] achieving up to 2 s/frame. In Jean-Louis Dillenseger, Soizic Laguitton, and Éric Delabrousse, Fast simulation of ultrasound images from a ct volume, Computers in biology and medicine, 39(2): 180-186, 2009 [DLD09], Dillenseger et al demonstrated a convolution-based simulation using multi-dimensional fractal filling and 2D anatomical maps that were segmented from CT images simply by thresholding.

Ray-based Generative Simulation (rUSim). This set of techniques “walk” in the image along the beam propagation axis determining the amount of reflected, attenuated, etc wave amplitudes at each image pixel point. Most methods in this category perform 2D planar simulation, which is often sliced in real-time from a 3D volumetric model. A realistic appearance of simulated ultrasonic speckle is essential for a plausible ultrasound ray-based generative simulation. An efficient and realistic model for ultrasonic speckle is the convolution of an ultrasound PSF with a parametrized distribution of point scatterers. A possible ultrasound speckle model as represented in 2D computes ultrasonic speckle intensity I(x,y) by convolving point-like scatterers in the tissue T(x,y) with the ultrasonic impulse response H(x,y), or the so-called PSF, i.e. (Eq. 1):

I(x,y)=T(x,y)*H(x,y)

where H(x,y) approximates the ideal sinc kernel.

H(x,y) may for instance be represented as a Gaussian distribution modulated with cosine in the axial direction y (Eq. 2):

$$H ( x , y ) = {e}^{\frac{x}{{σ}_{x}} + \frac{y}{{σ}_{y}}} cos ( 2 π fy )$$

In Benny Bürger, Sascha Bettinghausen, Matthias Radle, and Jiirgen Hesser, Real-time gpu-based ultrasound simulation using deformable mesh models, Medical Imaging, IEEE Transactions on, 32(3): 609-618, 2013 [BBRH13], to utilize GPU pipelines such as the Nvidia OptiX ray-tracing library (originally designed for computer-graphical rendering), Burger et al. proposed to use a discretized version of this model where scatterers are represented on a discretized texture grid, i.e. T[x,y]. They also introduced a 3-parameter approximation to model tissue-specific sparse scatterer patterns. This uses a normal distribution N(μ,σ), which has two parameters, and a scatterer sparsity parameter r, which is the ratio of texels populated with a scatterer. Such a model forms the basis of a ray-based simulation that also does “texture” look-ups with PSF-scatterer convolution patterns. This is demonstrated in [BBRH13] to yield excellent images for simulated phantoms. In comparison, however, the in-vivo image presented in that work is deficient in realism, potentially due to the following problems: (i) the scatterer distribution parameterizations of human tissues being sub-optimal, and (ii) the anatomical models not having been generated realistically; in contrast to their successful phantom images where the geometry is known accurately.

We observed that, for a given arbitrary tissue, such scatterer distributions that would generate a realistic image are not known a priori, and currently there is no principled method to extract such scatterer patterns for given target tissues to be simulated. There is therefore a need for a system and method to solve the inverse problem of estimating a scatterer distribution from sample ultrasound signals/images. If such distribution is obtained from a homogeneous tissue region, then a parametric model such as in [BBRH13] can be used to abstract a scatterer model. For instance, in the case of the simulation model of [BBRH13], a principled approach is needed to automatically identify μ, σ, and r from sample US images.

Furthermore, an efficient and realistic method is needed to automatically generate a diversity of scatterers that are suitable for a diversity of real-time ultrasound simulator implementations with similar end-user interactivity as encountered in real-world medical practice. The resulting scatterers may then be stored as a library of scatterers corresponding to different simulation practices. Such a library of scatterers may then be used in a diversity of ray-based ultrasound simulation applications with more realistic tissue appearance compared to state-of-the-art methods.

### BRIEF SUMMARY

A method and apparatus to reconstruct scatterers suitable for ultrasound imaging simulation are described, comprising: acquiring a plurality of ultrasound signal samples, each corresponding to a different ultrasound capture; estimating at least one Point Spread Function (PSF) associated with the plurality of ultrasound signal samples; reconstructing a scatterer representation from said plurality of ultrasound signal samples and said Point Spread Function.

The reconstructed scatterers may be directly registered into a scatterer library. In further embodiments, an additional step of modeling the scatterers may be applied. Statistical distribution parametrization or texture synthesis may be used to model the scatterers. The resulting scatterer model may then be registered into a scatterer library, to be referred to by a ray tracing ultrasound simulation system.

### BRIEF DESCRIPTION OF THE DRAWINGS

FIG. 1 represents a scatterer generator system.

FIG. 2 shows a homogeneous tissue sample region in an ultrasound B-mode image, corresponding to the impulse response of a superposition of microscopic, by itself unobservable scatterers.

FIG. 3A represents a PSF estimation system.

FIG. 3B shows a possible embodiment of the PSF estimation unit in a PSF estimation system.

FIG. 4 shows different PSFs estimated for four different axial and lateral ultrasound signal captures and the lateral spread of resulting beam profile according to one embodiment of the PSF estimation system.

FIG. 5 shows the scatterer map reconstruction from a complete ultrasound image of a liver according to one embodiment of the scatterer generator system.

FIG. 6 shows the reconstruction of four homogenous tissue regions from a US image of a pelvic phantom which can be used for simulation.

FIG. 7 shows three different statistical distribution model parametrizations corresponding to different scatterers.

FIG. 8 illustrates three different synthetized uterus image examples as may be simulated from scatterers as generated according to different possible embodiments of the invention, in comparison with the original ultrasound image.

FIG. 9 shows ultrasound images simulated with changing ultrasound parameters from scatterers as generated according to the proposed method.

FIG. 10 shows ultrasound images simulated for different view directions from the same volume of scatterers as generated according to the proposed method.

### DETAILED DESCRIPTION

### Scatterer Generator

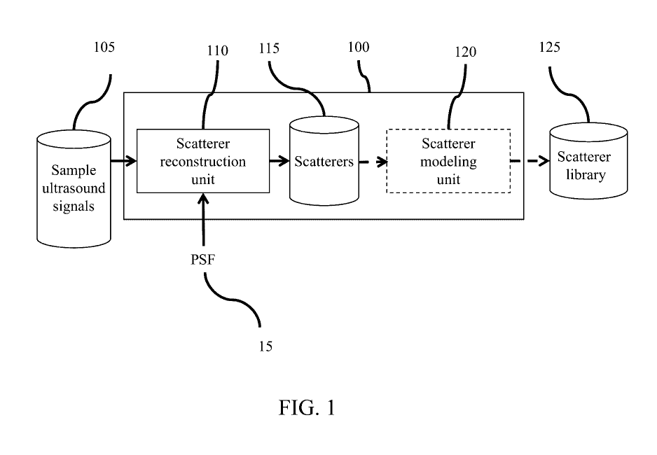

FIG. 1 represents a scatterer generator system 100 comprising a scatterer reconstruction unit 110 and a scatterer modeling unit 120. The scatterer reconstruction unit 110 may comprise at least one central processing unit (“CPU”) circuit, at least one memory, controlling modules, and communication modules to compute and record onto memory 115 the scatterers corresponding to sample ultrasound signals 105. The scatterer modeling unit 120 may comprise at least one central processing unit (“CPU”) circuit, at least one memory, controlling modules, and communication modules to compute and record onto memory 125 the library of scatterers corresponding to sample ultrasound signals 105. Different embodiments are also possible, e.g. an FPGA implementation of either the scatterer reconstruction unit 110 or the scatterer modeling unit 120 or both.

The scatterer modeling unit 120 and the scatterer reconstruction unit 110 may be physically the same or different data processing units. The scatterer modeling unit 120 may be adapted to the scatterer reconstruction unit 110, since depending on the quality of the scatterer reconstruction, more or less statistical parametrization operations may be needed. In a possible embodiment (not represented) the reconstructed scatterer representation (scatterer map) 115 may also be directly recorded onto the scatterer library memory 125 (one to one mapping parametrization).

### Ultrasound Signal Sample Acquisition

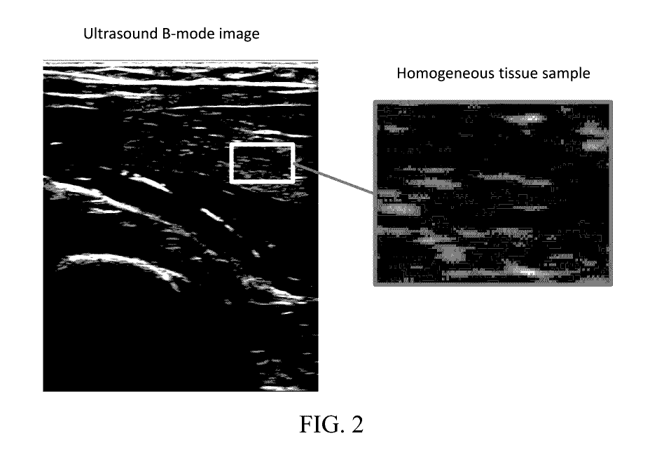

The scatterer generator 100 takes as input a plurality of sample ultrasound signals 105 corresponding to a real ultrasound capture and the associated Point Spread Function PSF 15. The scatterer generator 100 may use an acquisition unit (not represented) in connection with an ultrasound probe to acquire sample ultrasound signals 105 from a diversity of ultrasound probe settings, a diversity of view angles, and/or a diversity of tissue regions, corresponding to different speckle properties i.e. different scatterers 115. As an illustration of those different properties in real images, FIG. 2 shows an enlarged image from a homogeneous tissue sample in a B-mode ultrasound image capture, corresponding to the impulse response of a superposition of microscopic, by itself unobservable scatterers.

In order to improve the statistical robustness of the scatterer generator steps and in order to be able to synthesize images of different tissue types from actual ultrasound images to be used in simulations with arbitrary view angles and transducer settings, different captures from the same tissue regions may be used as input to the scatterer generator system.

Moreover, the sample ultrasound signals 105 may be generated in a medical practice environment from different ultrasound transmit/receive sequences, for instance by beam-steering, and/or different probe settings, for instance multiple acquisitions via focus, frequency, and other changes as done in compounding techniques, as known to those skilled in the art, in order to acquire patient-specific samples.

In a possible embodiment, the scatterer generator 100 first acquires, with an acquisition unit, a radiofrequency (RF) ultrasound signal, and applies a Hilbert transform to extract its envelope image signal without the carrier wave. Alternatively, sample ultrasound RF signals may be directly used as input to the scatterer reconstruction unit.

### PSF Sstimation

Using the envelope image, the corresponding PSF 15 may be mathematically modeled as a

Gaussian modulated cosine pulse (Eq.2):

$$H ( x , y ) = {e}^{\frac{x}{{σ}_{x}} + \frac{y}{{σ}_{y}}} cos ( 2 π fy )$$

In ultrasound the PSF is not constant over the whole domain, but changes with respect to position, predominantly and most importantly with respect to image depth. In other words, sigma _x and sigma_y vary across different locations in the image, where y is the depth axis. Hence, the PSF can be seen as a spatially varying function that returns a different PSF kernel H as a function of image position (or depth). The PSF as a function of image depth can also be interpreted as a description of a continuous ultrasound transducer beam profile, with a distinct beam profile for each transducer/settings. The PSF may be modeled in 2D or 3D depending on the needs of the application.

In a possible embodiment, the PSF function H(x,y) may be approximated experimentally from the size of the speckles in the images. In other possible embodiments, it may determined via simulations from accurate models of the transducer, such as for instance using the Fieldll simulation in accordance with the method described in J. Ng, R. Prager, N. Kingsbury, G. Treece, and A. Gee, Wavelet restoration of medical pulse-echo ultrasound images in an EM framework, IEEE TUFFC, 54(3):550-568, 2007 [Ng2007], as is well known to those skilled in the art. As known to those skilled in the art, the PSF function may also be determined by imaging sub-wavelength synthetic features, e.g. wires, in degassed water.

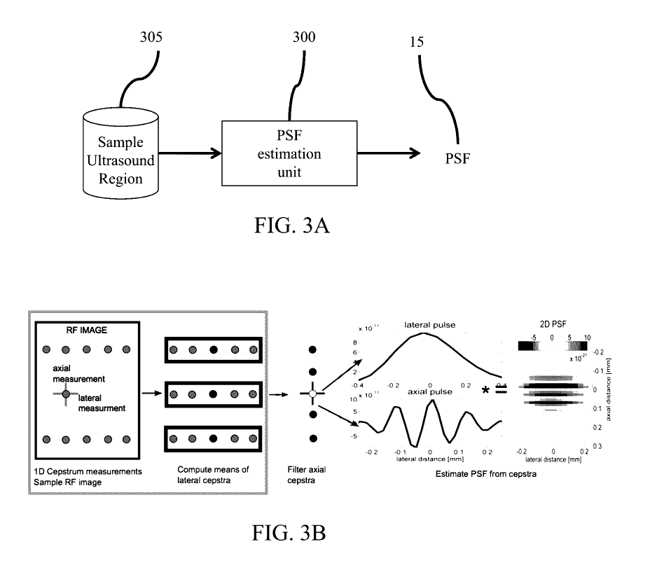

In a possible embodiment, represented in FIG. 3A, the Point Spread Function 15 may be directly estimated from input ultrasound image samples 305 by a PSF estimation unit 300. In a possible embodiment, a piecewise-constant PSF function may be estimated over a specific region of interest in the input samples 305. In an alternate possible embodiment, since the PSF mainly changes by depth, a variable PSF function H may be estimated as a smoothly varying function of image depth d over a full depth range of an image in the input samples 305, corresponding to one or multiple foci. In a possible embodiment, as will be apparent to those skilled in the art, homomorphic filtering in the cepstrum domain may be used to estimate the PSF. In a possible embodiment, a separable PSF may be estimated by applying the homomorphic filtering separately in the axial and lateral direction, using more robust 1D phase unwrapping in either direction. In a possible embodiment, represented in FIG. 3B, the PSF estimation unit 300 may then:
receive 1D cepstrum measurements respectively in the axial and lateral directions in the sample RF image;
compute the arithmetic means of lateral cepstra, for each axial value;
filter the resulting axial cepstra, with homomorphic filtering;
estimate the PSF from the cepstra, as a function of axial and lateral distances respectively.

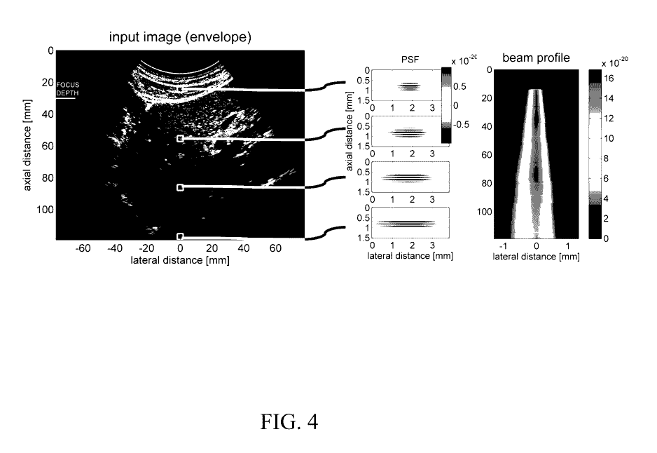

This provides a depth-dependent PSF function (beam profile) where the arithmetic mean provides increases signal-to-noise of estimates by averaging several PSF observations at the same depth. As an illustration, FIG. 4 shows on the left an envelope image of a liver scan acquired with a convex probe. FIG. 4 shows in the middle four samples of the estimated PSF at four different depths, according to one embodiment of the PSF estimation system. As expected for a convex probe, the resulting beam profile, the depth-dependent lateral spread of which is represented on the right, exhibits an almost linear increase of the beam width. The resulting estimated PSF 15 can thus be used as input to the scatterer reconstruction unit 110 in the scatterer generator system 100 of FIG. 1, as will now be described in further detail.

### Scatterer Reconstruction

Without loss of generality, we describe an embodiment of scatterer reconstruction which is defined in 2D (for lateral and axial directions). Note that these definitions can be extended to 3D by further considering an elevation direction. Once a sample ultrasound image homogeneous region has been acquired and using the estimated PSF 15, the scatterer representation T(x,y) (sactterer map) 115 may be reconstructed based on Eq. 1 for given speckle image appearances. Convolution can be written as a linear operation Ax=b. A is then the convolution matrix, each row of which contains all PSF values at columns corresponding to image indices that this PSF kernel would operate on for that row. x is the column vector of all scatterers discretized on a grid, b is the resulting column vector of image appearances. For an overall number of n scatterers and a number of US image pixels m, x has then n elements, b has m elements, and A is an n×m matrix (note that this holds for both 2D and 3D).

Since H(x,y) may be approximated as a separable function, the convolution matrix may be written as a product A=CD of Toeplitz matrices, where C and D represent the convolution in lateral and axial directions, respectively. In one embodiment, assuming the PSF may be approximated by a Gaussian model, the PSF convolution kernel may be cutoff at 4 standard deviations where the energy becomes negligible. For example, in one of our experimentations with 5 MHz ultrasound center frequency, we used two wavelengths in the axial direction and 3 wavelengths in the lateral direction, respectively, which resulted in a window size of 20 and 12 pixels for the lateral and axial convolution kernels (measured in ultrasound image scale), and A had accordingly at most 240 nonzero entries per row. Empirical tests with scatterer resolution of the size of the image and up to 16 times its resolution gave us satisfactory results also with reasonable computation times of seconds to minutes for scatterer reconstruction.

Note that if the number of measurements is equal or less than the number of unknowns, e.g. while using a single image instead of multiple images to reconstruct the scatterers at a higher resolution, or when the observations are not linearly independent, then the inverse problem may be under-determined and hence regularization or additional constraints may be required to get viable solutions. In a possible embodiment, to obtain sparse scatterer reconstructions, a total-variation regularization formulation using L₁-norm may be solved as follows (Eq. 4):

$$\hat{x} = arg {min}_{x} {‖ Ax - b ‖}_{2} + δ {‖ x ‖}_{1} s . t . x \geq 0$$

This formulation favors positive sparse x with small scatterer amplitudes. The constraint ensures the scatterer responses to be positive, modeling the actual physics while also increasing solution robustness. In other possible embodiments, other regularization norms may be used, for instance the L2 norm, the L2L1 Lasso formulation, the L1L1 formulation, or other formulations, as known to those skilled in the art. Various mathematical solvers may also be used, for instance the Alternating Direction Method of Multipliers (ADDM), the Interior Point method, or the YALL1 method, as known to those skilled in the art of solving optimization problems.

### Optimization for Large Ultrasound Volume Acquisition

As will be apparent to those skilled in the art, attempting to solve for a full-sized US image with possibly multiple measurements in a single linear system may exceed the memory and computing capacities of some embodiments of the scatterer reconstruction unit 110. In a possible embodiment, the scatterer reconstruction unit 110 may thus partition the ultrasound image into smaller blocks and may solve the scatterer reconstruction inverse problem individually for each block instead of the full image. The scatterer reconstruction unit 110 may then combine the resulting individual solutions into a single reconstructed scatterer representation map. In a possible embodiment, the scatterer reconstruction unit 110 may enforce proper boundary conditions to avoid discontinuities at the seams between adjacent blocks. In an alternative embodiment, the scatterer reconstruction unit 110 may subtract the speckle contribution of the previously computed scatterers from the RF measurements of adjacent blocks, which can also be seen as constraining the scatterers in the border region to match the previously computed ones. In a possible embodiment, the scatterer reconstruction unit 110 selects a border region that is large enough to capture at least half the footprint of the PSF, to ensure that all observations overlapping the current block can be explained by scatterers within the block. Other embodiments are also possible.

### Raw Reconstructed Scatterer Registration

The scatterer representation T[x,y] (scatterer map 115) may be reconstructed thanks to the above methods, and used in ray-tracing either directly or through an additional modeling step 120, as will now be described in further detail.

In one embodiment, the raw reconstructed scatterers 115 may be reconstructed for the entire images, for instance by solving fully Eq. 1 or by combining several smaller reconstructions thereof In a possible embodiment, the scatterer reconstruction unit 110 first solves the inverse problem in the polar coordinate frame of the original RF signal lines, then applies a scan conversion into the cartesian domain to generate the scatterer representation 115. Other embodiments are also possible.

The raw reconstructed scatterers may subsequently be directly registered in a scatterer library 125. This embodiment allows for the imaging simulation of exactly the originally imaged anatomical location, whereas the ultrasound imaging location or parameters can be modified. For instance, the same imaged patient may be viewed from different locations or with different probe settings.

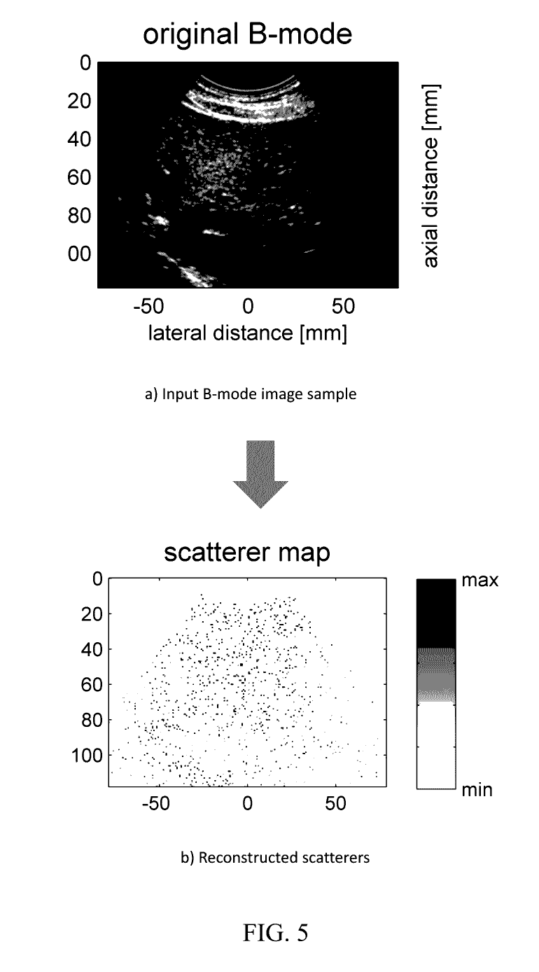

As an illustration, FIG. 5 shows an example of a scattterer map reconstruction 115 from a clinical liver ultrasound image 105. FIG. 5a) shows a B-mode visualization of the input image, acquired with a UltraSonix 4DC7 3/40 convex probe operating at 4.5 MHz, with a sampling frequency 20 MHz and a field-of-view of 75°. The input RF image has a resolution of 3136*192 with an axial spacing of 0.0385 mm and is partitioned in 36 smaller blocks to optimize the inverse problem resolution by the scatterer reconstruction unit 110. FIG. 5b) shows the resulting scatterer map generated by the scatterer reconstruction unit 110 from said RF image, while a resolution of 3136*1920, downsampled by a factor of 50 for visualization in FIG. 5b).

### Scatterer Modeling

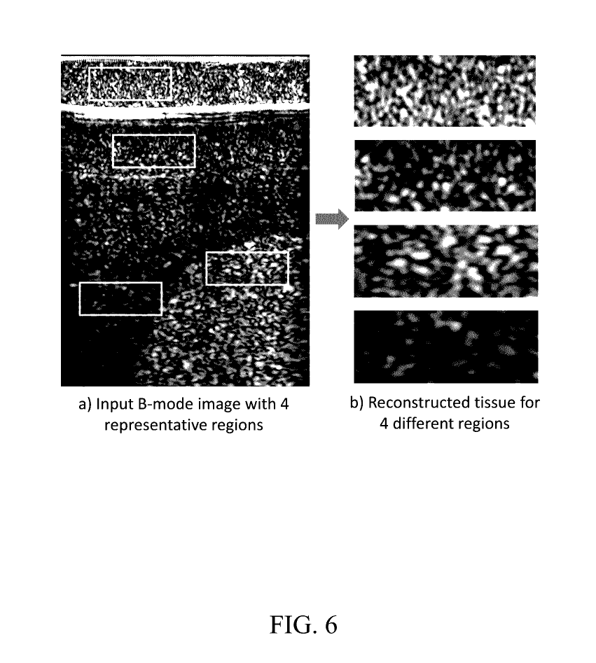

In another embodiment, the scatterer generator of FIG. 1 may further comprise a modeling unit 120 to compute and register into a scatterer library 120 a more compact representation 125 of the reconstructed scatterers 115. This more compact model may facilitate a more efficient use of the reconstructed scatterer representations by a real-time ultrasound simulation system to populate arbitrary ultrasound geometries and shapes. As illustrated in FIG. 6a), in actual ultrasound practice, different anatomy regions correspond to different scatterers. A virtual reality or augmented reality (VR/AR) ultrasound simulator such as an adaptation to ultrasound imaging of the endoscopy simulator described in U.S. Pat. No. 8,992,230 may comprise a virtual anatomy model with spatial divisions into separate anatomical regions (so-called segmentations) for which the ultrasound texture (speckle) appearance differ so-called homogeneous regions. Then a different scatterer instantiation may be used for each of those regions. This may be performed in 3D by computing the instantiation offline and storing it for online image simulation use as in [BBRH13]. Before image simulation, such 3D scatterer instantiations may also be deformed together with virtual-reality simulated tissue deformations, allowing for deformable model simulations for added realism. Alternatively, for each 2D image to be simulated, a slice may be extracted from a given segmentation and a 2D scatterer instantiation may then be performed in that slice. The modeling unit 120 may take one or more representative samples from the input ultrasound image and derive the reconstructed scatterers as different scatterer models and register them into the scatterer library 125. FIG. 6a) shows the representative samples as gray boxes, and FIG. 6b) shows the reconstructed tissues from these samples using four different scatterer models.

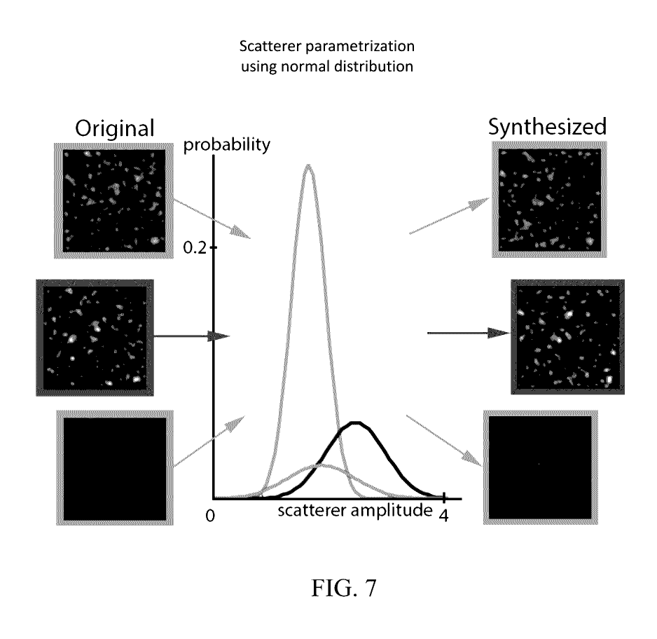

In a first possible embodiment, assuming a statistical model as described in [BBRH13], in the case of a simple normal distribution model, the three parameters y, a, and r (mean, standard deviation, and sparsity) may be estimated simply as follows. The number of non-zero scatterer texels using a threshold epsilon gives the ratio r. The mean and the standard deviation of these non-zero scatterers then yield μ and σ, respectively. FIG. 7 illustrates the resulting simulation of three different tissue appearances, corresponding to three different sets of parameters (μ, σ, r).

When instantiating a new scatterer texture, one should note that negative-amplitude scatterer, which are physically not possible, may be generated by the above normal distribution. Depending on how these scatterers with negative amplitudes are treated, the final scatterer statistics may therefore slightly change. In one embodiment, scatterer amplitudes may be clamped to zero, hence slightly lowering the actual value ofr in the synthesized texture. Other embodiments are also possible. The above parametrization step has been described assuming a statistical model as described in

[BBRH13]. The parameters may be estimated from the reconstructed scatterer texture by a typical maximum-likelihood estimation step. This simple embodiment of a statistical model works best for almost homogenous tissue but it may not, optimally capture certain structural features. The proposed method is, however, not limited to this statistical model. For instance, in the case of more general statistical models, the parametrization step may be done using methods, such as expectation-maximization techniques, known to those skilled in the art.

Other non-statistical texture synthesis models may also be used. For instance, in a possible embodiment, methods of texture synthesis may be used. In this embodiment, we use a method proposed by Michael Ashikhmin in “Synthesizing Natural Textures”, Proceedings of I3D, 217-226, 2001 [A01], but many alternatives exist. Such methods assemble larger, non-replicating images from smaller examples, to distribute the scatterers, at the expense of a less compact representation than with the statistical parametrization embodiment, but they enable to capture more structure and variations of the input tissue.

### Scatterer Library

Depending on the actual embodiment as described above, with reference to FIG. 1, either the raw reconstructed scatterers 115 or the scatterer model parameters out from the modeling unit 120 according to various possible modeling embodiments may be registered by the scatterer generator unit 100 as an entry to the scatterer library 125.

### Experimental Results

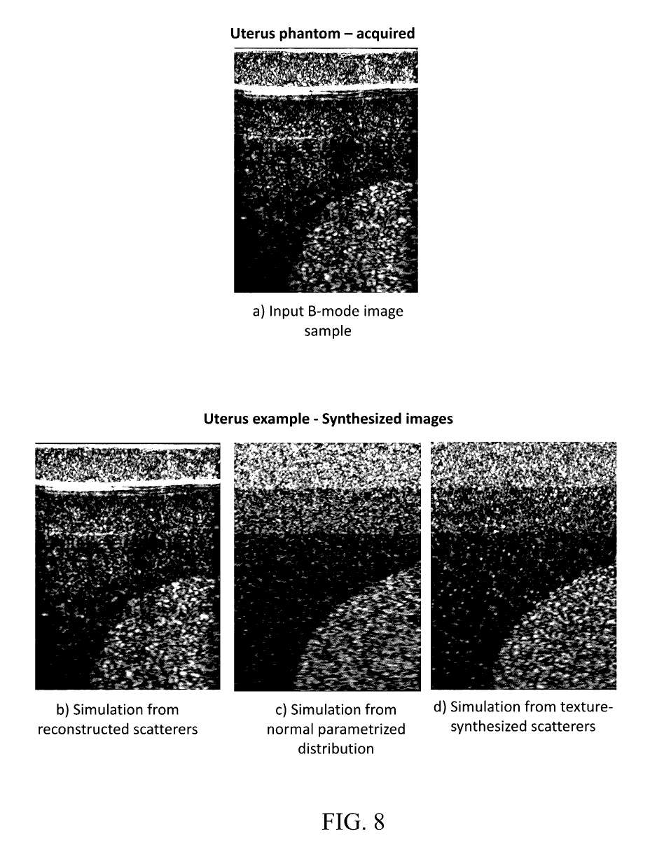

The scatterer library may then be referred to by an actual ultrasound imaging simulator to create speckle images of varying shape and resolution (e.g., a 2D image or a 3D volumetric texture). For the sake of illustration, FIG. 8 shows a pelvic phantom (FIG. 8a) image, and the results from three different possible embodiments of using respectively the raw reconstructed scatterers embodiment (FIG. 8b), the normal distribution model parametrization embodiment (FIG. 8c) and the texture-synthesis embodiment (FIG. 8d) in an ultrasound simulation experiment where convolution with a PSF of the same ultrasound beam profile was used for both reconstruction and simulation results.

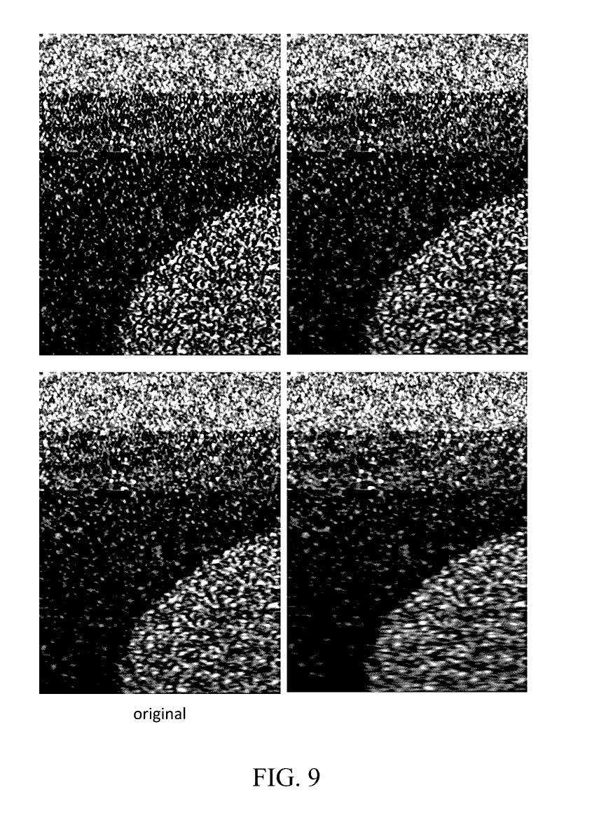

FIG. 9 demonstrates the flexibility of the reconstructed scatterers under varying ultrasound parameters, in this case a shifted ultrasound beam focus, ordered by decreasing depth values from top left to bottom right, when used in an ultrasound simulation experiment.

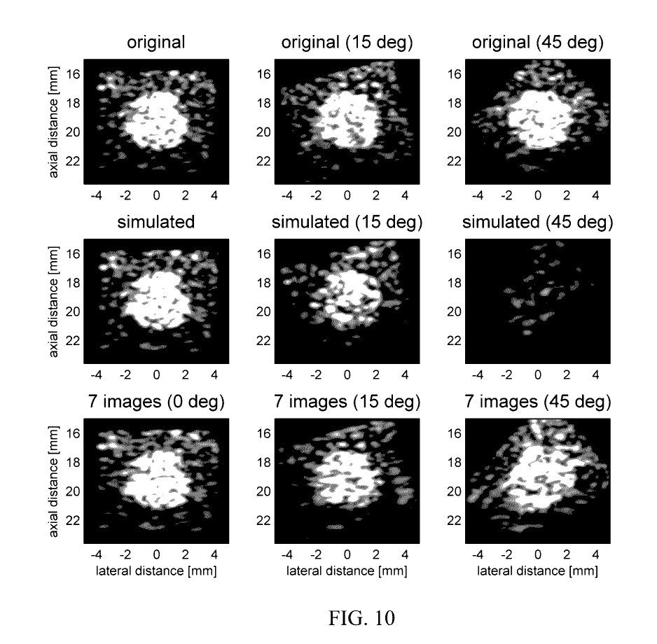

FIG. 10 further shows the resulting ultrasound simulation respectively from one input ultrasound image and seven input images when viewing the scatterer volume from different directions (i.e., rotating the US transducer direction by 15°, and 45°, respectively).

### Other Embodiments and Applications

Although the detailed description above contains many specificities, these should not be construed as limiting the scope of the embodiments but as merely providing illustrations of some of several embodiments.

While exemplary scatterer extraction and modeling detailed embodiments have been described in 2D for the sake of simpler mathematical representation, the actual tissue scatterers reside in a 3D domain. To generalize the embodiments to 3D, either 3D distributions may be approximated from 2D observations, or from having collected a 3D ultrasound volume (or equivalently several spatially-registered 2D images) in the domain. A 3D scatterer distribution T[x,y,z] may be reconstructed by the scatterer reconstruction unit 110 by considering a 3D PSF and a 3D scatterer distribution in Eq 1, also assuming a 3D convolution operation. Aside from 3D ultrasound probes having the ability to acquire 3D image volumes or several 2D images e.g. in a fan shape, such spatially-aligned 2D images may also be collected using position (e.g. magnetically or optically) tracked transducers as well as by applying compressions/deformations on the tissue.

Other applications of the proposed scatterer model generator are also possible beyond ray-based simulation methods, such as for instance tissue inpainting for image-based ultrasound simulation methods, as known to those skilled in the art. The reconstructed scatterer distributions or their statistical models thereof may also potentially contain discriminant or diagnostic information about the underlying imaged tissues, that may be used in medical training applications.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments.

As will be apparent to those skilled in the art of digital data communications, the methods described herein may be indifferently applied to various data structures such as data files or data streams. The terms “data”, “data structures”, “data fields”, “file”, or “stream” may thus be used indifferently throughout this specification.

Although the detailed description above contains many specific details, these should not be construed as limiting the scope of the embodiments but as merely providing illustrations of some of several embodiments.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments.

In addition, it should be understood that any figures which highlight the functionality and advantages are presented for example purposes only. The disclosed methods are sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms. Modules may constitute either software modules (e.g., code embodied on a machine-readable medium or in a transmission signal) or hardware modules. A “hardware module” is a tangible unit capable of performing certain operations and may be configured or arranged in a certain physical manner. In various example embodiments, one or more computer systems (e.g., a standalone computer system, a client computer system, or a server computer system) or one or more hardware modules of a computer system (e.g., a processor or a group of processors) may be configured by software (e.g., an application or application portion) as a hardware module that operates to perform certain operations as described herein.

In some embodiments, a hardware module may be implemented mechanically, electronically, or any suitable combination thereof For example, a hardware module may include dedicated circuitry or logic that is permanently configured to perform certain operations. For example, a hardware module may be a special-purpose processor, such as a field-programmable gate array (FPGA) or an ASIC. A hardware module may also include programmable logic or circuitry that is temporarily configured by software to perform certain operations. For example, a hardware module may include software encompassed within a general-purpose processor or other programmable processor. It will be appreciated that the decision to implement a hardware module mechanically, in dedicated and permanently configured circuitry, or in temporarily configured circuitry (e.g., configured by software) may be driven by cost and time considerations.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module implemented using one or more processors.

Similarly, the methods described herein may be at least partially processor-implemented, a processor being an example of hardware. For example, at least some of the operations of a method may be performed by one or more processors or processor-implemented modules.

Some portions of the subject matter discussed herein may be presented in terms of algorithms or symbolic representations of operations on data stored as bits or binary digital signals within a machine memory (e.g., a computer memory). Such algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities.

Although an overview of the inventive subject matter has been described with reference to specific example embodiments, various modifications and changes may be made to these embodiments without departing from the broader spirit and scope of embodiments of the present invention. For example, various embodiments or features thereof may be mixed and matched or made optional by a person of ordinary skill in the art. Such embodiments of the inventive subject matter may be referred to herein, individually or collectively, by the term “invention” merely for convenience and without intending to voluntarily limit the scope of this application to any single invention or inventive concept if more than one is, in fact, disclosed.

The embodiments illustrated herein are believed to be described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present invention. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present invention as represented by the appended claims. The specification and drawings are, accordingly, to be regarded in an illustrative rather than a restrictive sense.

Finally, it is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A method for generating, with at least one processor, a scatterer representation for ultrasound imaging simulation, comprising:
acquiring a plurality of ultrasound signal samples, each corresponding to a different ultrasound capture;
estimating at least one point spread function (PSF) associated with at least one ultrasound capture; and
reconstructing a scatterer representation from said plurality of ultrasound signal samples and said PSF.

2. The method of claim 1, wherein the scatterer representation is a 2D matrix T[x,y] or a 3D matrix T[x,y,z].

3. The method of claim 1 , wherein the plurality of ultrasound samples is generated from different locations or viewing angles.

4. The method of claim 1, wherein the plurality of ultrasound samples is generated from different ultrasound probe settings.

5. The method of claim 1, wherein the plurality of ultrasound samples is generated from different ultrasound transmit/receive sequences when interacting with a homogeneous tissue region.

6. The method of claim 1, wherein the plurality of ultrasound samples is generated by beam-steering.

7. The method of claim 1, wherein the plurality of ultrasound samples is generated from different anatomical deformations.

8. The method of claim 7, wherein different anatomical deformations are generated by manipulating the ultrasound probe.

9. The method of claim 1, wherein estimating the PSF associated with at least one ultrasound capture comprises a step of homomorphic filtering in the cepstrum domain.

10. The method of claim 9, wherein homomorphic filtering is applied separately in the axial and the lateral directions.

11. The method of claim 1, further comprising registering the scatterer representation in a scatterer library.

12. The method of claim 1, further comprising modeling the scatterer representation into a scatterer model.

13. The method of claim 12, wherein statistical distribution parameterization is used to model the scatterer representation.

14. The method of claim 13, wherein the statistical distribution parameterization is a normal distribution N(μ, σ) combined with a scatterer sparsity parameter r as the ratio of texels populated with said scatterer representation.

15. The method of claim 14, wherein texture synthesis is used to model the scatterer representation.

16. A system for generating a scatterer representation for ultrasound imaging simulation, comprising a scatterer reconstruction unit and a PSF estimation unit, the system being configured to:
acquire a plurality of ultrasound signal samples, each corresponding to a different ultrasound capture;
estimate at least one PSF associated with at least one ultrasound capture; and
reconstruct the scatterer representation from said plurality of ultrasound signal samples and said PSF.

17. The system of claim 16, wherein the PSF estimation unit is further configured to estimate the PSF associated with at least one ultrasound capture using a homomorphic filtering in the cepstrum domain.

18. Thesystem of claim 16, further comprising a scatterer modeling unit configured to model the scatterer representation into a scatterer model.

19. The system of claim 18, wherein statistical distribution parameterization is used by the scatterer modeling unit to model the scatterer representation.

20. The system of claim 18, wherein texture synthesis is used by the scatterer modeling unit to model the scatterer representation.
